(19) United States
(12) Patent Application Publication
(10) Pub. No.: US 2024/0016530 A1
(43) Pub. Date: Jan. 18, 2024

(54) BONE REDUCTION KIT

(71) Applicants: Turlough O'DONNELL, Dublin (IE); Malcolm O'KELLY, Dublin (IE)

(72) Inventors: Turlough O'DONNELL, Dublin (IE); Malcolm O'KELLY, Dublin (IE)

(21) Appl. No.: 18/256,224

(22) PCT Filed: Dec. 8, 2021
(86) PCT No.: PCT/EP2021/084867

§ 371 Date: Jun. 7, 2023

(30) Foreign Application Priority Data
Dec. 8, 2020 (GB) 2019316.5

(51) Int. Cl. A61B 17/80

(52) U.S. Cl. CPC A61B 17/808; A61B 17/8057; A61B 17/8061; A61B 2017/00862

(57) ABSTRACT

A bone reduction kit to hold a bone fixation plate (20) in position on an at least party reduced bone (16) while the bone fixation plate is fixed to the bone is described. The kit comprises a bone fixation plate (20) having a bone abutting surface and an obverse face (21) and a plurality of screw-receiving though-holes and a bone reduction forceps (1). The forceps comprises a first arm (2) having a proximal part (5) comprising a handle (6A) and a bifurcated distal part having a first fork (8A) with a first distal foot (9A) and a second fork (8B) with a second distal foot (9B), and a second arm (3) having a proximal part (10) comprising a handle (6B) and a distal part (11) comprising a bone engaging jaw (13) configured to engage a second surface (24) of the bone (16). The second arm is pivotally attached to the first arm by a first joint (4) disposed between the respective proximal and distal parts of the first and second arms. The obverse face (21) of the bone fixation plate (20) comprises two spaced-apart depressions (22) positioned and dimensioned to mate with first and second distal feet (9A, 9B) of the forceps whereby when the forceps is clamped on the partly reduced bone and plate, lateral movement of the plate with respect to the bone is prevented.

### FIELD OF THE INVENTION

The present invention relates to a bone reduction kit. The invention also relates to a method of plating a fractured bone that employs a bone reduction kit.

BACKGROUND TO THE INVENTION

The clavicle or collarbone is a long bone that serves as a strut between the shoulder blade and the sternum. Humans have two clavicles, left and right. It is the most commonly fractured bone in the body, accounting for 5% of all bone fractures. Approximately 17,000 clavicle fractures are reported daily on a global basis. The clavicle can be fractured due to direct impact on the bone, or due to impact to the shoulder from the force of falling on outstretched arm. When viewed from the front (anterior view), the bone has a generally straight appearance, and when viewed from above (superior view) it has a two-curve configuration from the sternal end to the acromial end, a so-called “lazy S” shape. About 5% of clavicle fractures occur at the sternal end (medial fractures), 10-15% occur at the acromial end (lateral fractures), and the vast majority of fractures occur in the midshaft (80-85%). Treatment of clavicle fractures include conservative treatments (treatment without surgery). This is a successful treatment for undisplaced 2-part clavicle fractures. However, if the fracture is significantly displaced or comminuted (in more than 2 pieces), conservative treatment results in a higher incidence of non-union or malunion of the fracture, which can cause significant persistent weakness and disability even if the fracture heals. It is generally accepted that surgical treatment of fractures is indicated for comminuted fractures, widely displaced and shortened fractures, segmental fractures and “Z-type” fractures. The most common surgical treatment for serious clavicle fractures involves use of superior plate fixation or anteroinferior plate fixation. These are monoplanar plates having a series of holes for receipt of bone fixation screws. In order to have the required strength to resist torsional and bending stiffness, these plates have to have a thickness of at least 4 mm along their length, which is quite large for an implant in this area of the skeleton, and uncomfortable for the patient. In addition, it is possible to use a percutaneous intramedullary screw, which has gained some popularity in recent years.

In the case of comminuted fractures, segmental fractures and Z-type fractures, it is extremely difficult to reduce the bone fragments and fix them in place with a plate, and the fragments need to be reduced point-to-point while the fixation wires or screws are fixed in place. The deforming forces of the muscles often prevent the reduction holding in place, and it can be extremely difficult to fix the plate to the bone before the fracture displaces. Superior plate fixation is most commonly utilized because it makes the bone fragment reduction a little easier, but the plates tend to be weak in bending stiffness, and do not allow early mobilization of the shoulder, as there is a significant risk of the plate bending and the fixation failing. In addition, the plate is quite superficial and usually palpable through the skin. It is not uncommon for the patient to request removal of the plate once the fracture has healed.

Anteroinferior plate fixation provides better bending stiffness, as it increases the area moment of inertia, but it is extremely technically difficult to accurately reduce clavicle bone fractures using anterior plates. In addition, it is impossible to reduce the comminuted fragments once the plate has been applied. Anteroinferior plates also contribute to devascularisation of the bone at the fracture site. Intramedullary screw or pin fixation is also technically challenging, and it is particularly difficult to reduce the fracture. It is unsatisfactory in fixing comminuted fragments, as it does not provide adequate stability at the fracture site. Indeed, due to the difficulty of both superior and anteroinferior plate fixation, as well as intramedullary screw fixation, many orthopaedic surgeons elect a conservative, non-surgical treatment, which commonly lead to non-union or malunion. This has a detrimental effect on long-term shoulder girdle function.

As described above, plating a clavicular fracture is a difficult procedure that requires the bone fragments to be reduced and then a plate fixed to the reduced bone while maintaining the bone in the reduced position. Bone reduction forceps may be employed during this procedure, but conventional bone reduction forceps tend to be 2-point forceps that do not securely grasp the bone during a plating procedure which can result in bone fragment displacement during the procedure leading mal-union of the fracture and failed treatment.

It is an object of the invention to overcome at least one of the above-referenced problems.

It is a particular object of the invention to provide a bone reduction forceps that can hold the reduced bone and fixation plate in a secure engagement during the fixing of the plate to the bone and prevent lateral movement of the plate with respect to the bone.

### SUMMARY OF THE INVENTION

In a first aspect, the invention provides a bone reduction kit to hold a bone fixation plate in position on an at least party (or fully) reduced bone while the bone fixation plate is fixed to the bone, comprising:
a bone fixation plate having a bone abutting surface and an obverse face and a plurality of screw-receiving though-holes; and
a bone reduction forceps comprising:
   a first arm having a proximal part comprising a handle and a bifurcated distal part having a first fork with a first distal foot and a second fork with a second distal foot;
   a second arm having a proximal part comprising a handle and a distal part comprising a bone engaging jaw configured to engage a second surface of the fractured bone to clamp the bone between the first and second arms;
   wherein the second arm is pivotally attached to the first arm by a first joint disposed between the respective proximal and distal parts of the first and second arms.

Generally, the obverse face of the bone fixation plate comprises two spaced-apart depressions positioned and dimensioned to mate with first and second distal feet of the forceps whereby when the forceps is clamped on the partly reduced bone and plate, lateral movement of the plate with respect to the bone is prevented.

The provision of a bone reduction forceps with a bifurcated first arm with two distal feet, and a fixing plate with spaced apart depressions on the obverse face dimensioned to mate with the distal feet of the forceps, provides a mechanism for the plate to be securely fixed in position on the bone without any significant lateral movement of the plate with respect to the bone during a plating operation.

In any embodiment, the depressions are disposed in a central part of the obverse face of the plate, for example, in a central third of the obverse face of the plate as illustrated in FIG. 3.

In any embodiment, the first and second distal feet are disposed at a distal end of the first and second forks.

In any embodiment, the first and second distal feet comprises a resiliently deformable elastomeric material.

In any embodiment, the first and second distal feet comprises a rubber material.

In any embodiment, at least one or both of the depressions is a screw-receiving through-hole. The screw receiving hole may be countersunk or counterbored, in which the depression is provided by the countersunk or counterbored upper part of the screw hole.

In any embodiment, the first and second distal feet are dimensioned to nest snugly within the respective depressions. In any embodiment, each of the first and second distal feet taper inwardly towards a distal tip of the feet (e.g. frustrum shaped feet). In any embodiment, the depressions taper inwardly (e.g. frustrum shaped depression). In any embodiment, a sidewall of the or each depression may be chamfered, typically at a chamfer angle of about 60°-120°.

In any embodiment, one or both of the first and second distal feet has a frusto-conical shape.

In any embodiment, the depressions have a frusto-conical shape that optionally match the frusto-conical shape of the respective first and second distal feet.

In any embodiment, the first distal foot and second distal foot are laterally spaced apart by about 2-10 cm.

In any embodiment, the depression (for example the counter-sunk or counter-bored hole may have a diameter of about 0.5 to 3.0 cm, 0.5 to 2.0 cm, or 0.5 to 1.5 cm.

In any embodiment, the bone engaging jaw is counter-opposed to a position in between the first and second distal feet.

In any embodiment, the bone engaging jaw comprises a plurality of teeth.

In any embodiment, the forceps comprises a ratcheting mechanism attached to one of the first and second arms. The ratcheting mechanism may comprise a ratcheting rack attached to one of the arms.

In any embodiment, the bone fixation plate is a clavicle fixation plate typically configured to conform to a superior or inferior surface of a reduced human clavicle.

In any embodiment, the second arm is bifurcated and comprises two forks and two bone-engaging jaws.

In another aspect, the invention provides a method of plating a fractured bone that employs a bone reduction kit according to the invention, the method comprising the steps of:
at least partly reducing the bone fragments;
applying the bone fixation plate to a surface of the at least party reduced bone;
aligning the bone reduction forceps with the bone fixation plate and bone such that the first and second distal feet of the forceps are disposed above the depressions in the obverse face of the plate and the bone engaging jaw is disposed adjacent the second surface of the bone;
actuating the bone reduction forceps to clamp the bone fixation plate to the at least partly reduced bone with the first and second distal feet securely mated with the respective depressions;
fixing the bone fixation plate to the bone while the bone reduction forceps holds the bone and plate secured together; and
releasing and removing the bone reduction forceps from the bone.

In any embodiment, the bone is a clavicle and the bone fixation plate is a clavicle fixation plate.

In any embodiment, the fracture is a comminuted mid-shaft fracture of the clavicle.

In another aspect, the invention provides a bone reduction forceps comprising:
a first arm having a proximal part comprising a handle and a bifurcated distal part having a first fork with a first distal foot and a second fork with a second distal foot;
a second arm having a proximal part comprising a handle and a distal part comprising a bone engaging jaw configured to engage a second surface of the fractured bone to clamp the bone between the first and second arms;
wherein the second arm is pivotally attached to the first arm by a first joint disposed between the respective proximal and distal parts of the first and second arms.

In any embodiment, the first and second distal feet comprises a resiliently deformable elastomeric material.

In any embodiment, the first and second distal feet comprise a rubber material.

In any embodiment, the first and second distal feel are configured to mate with, respectively, a countersunk or counterbored hole provided on an obverse face of a bone fixing plate. The hole may be a through hole, for example a screw hole.

In any embodiment, one or both of the first and second distal feet has a frusto-conical shape.

In any embodiment, the first distal foot and second distal foot are laterally spaced apart by about 2-10 cm.

In any embodiment, the bone engaging jaw is counter-opposed to a position in between the first and second distal feet.

In any embodiment, the bone engaging jaw comprises a plurality of teeth.

In any embodiment, the second arm is bifurcated and comprises two forks and two bone-engaging jaws.

In any embodiment, the forceps comprises a ratcheting mechanism attached to one of the first and second arms. The ratcheting mechanism may comprise a ratcheting rack attached to one of the arms.

Other aspects and preferred embodiments of the invention are defined and described in the other claims set out below.

### BRIEF DESCRIPTION OF THE FIGURES

FIG. 1 is a side elevational view of a bone reduction forceps forming part of a kit according to the invention shown from distal (right hand side) to proximal (left hand side).

FIG. 2 is a front elevational view (looking in distal to proximal direction) of the bone reduction forceps of FIG. 1.

FIG. 3 is a sectional elevational view (looking in distal to proximal direction) of the bone reduction forceps of FIG. 2 shown holding a clavicle fixation plate to a superior surface of a fractured clavicle bone.

FIG. 4 is a side elevation view of the bone reduction forceps similar to FIG. 1.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

The term “bifurcated” as applied to the distal part of the first (or second) arm means that the arm forks into two forks at a forking point. Generally, the forks diverge in a symmetrical manner. Typically, the forks are mirror images of each other. Although the embodiment described herein, show the first arm having a bifurcated distal part, it will be appreciated that the second arm may also bifurcate Likewise, the invention is not restricted to first and/or second arms that are bifurcated, but may be embodied with an arm having a distal part that forks into three or more arms, depending on the application.

The term “bone fixation plate” refers to a plate used in orthopaedic surgery to attach to a fractured bone to provide structural support to the bone, keep the bone in an anatomically reduced position, and aid in the healing process. One example of a bone fixation plate is a dynamic compression plate. Generally, bone fixation plates include a number of holes that allow the plate to the fixed to the bone with screws. Often the holes are countersunk holes. The plate may be contoured to the shape of a specific bone. Generally, the plate is monoplanar. The plate comprises depressions dimensioned to receive first and second feet of the first arm of the forceps. The or each depression may form the upper part of a countersunk hole in the plate. The or each depression may extend fully or only partially through the plate. The or each depression may have an inwardly tapering sidewall. The or each depression, or a part thereof, is generally dimensioned to allow the feet and depressions mate snugly. This means that they nest together in a closely fitting arrangement with little play, which helps prevent movement of the plate relative to the feet when the forceps is engaged with the bone and the plate.

The term “handle” refers to formations on the proximal end of each arm, for example finger or palm engaging loop or handle that facilitate a surgeon holding and using the forceps.

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, camels, bison, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human. As used herein, the term “equine” refers to mammals of the family Equidae, which includes horses, donkeys, asses, kiang and zebra.

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

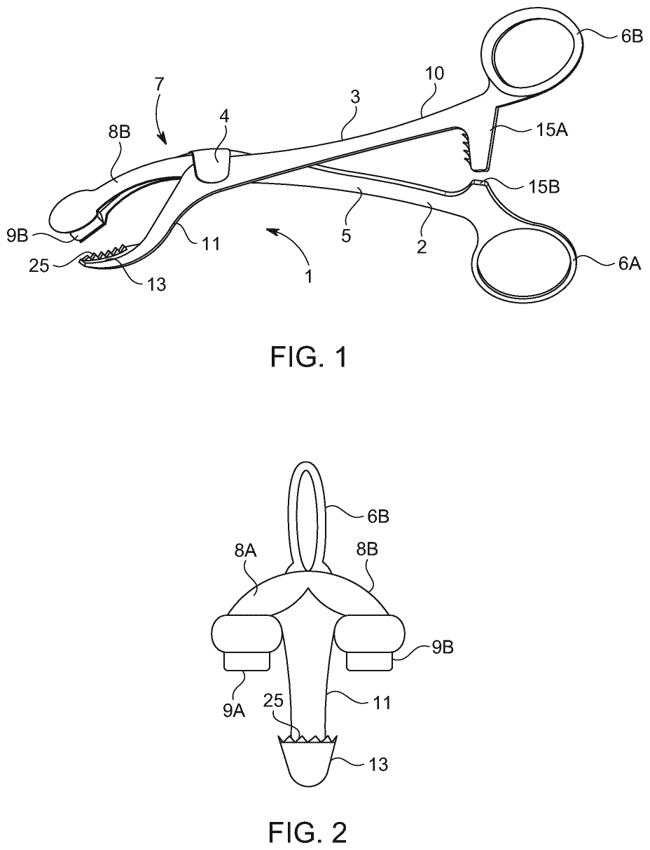

Referring to the drawings, and initially to FIGS. 1 and 2, a bone reduction forceps according to the invention is described, indicated generally by the reference numeral 1. The forceps comprises a first arm 2, second arm 3, and a pivot joint 4 providing pivoting scissors-like articulation of the arms.

The first arm 2 has a proximal section 5 with a handle 6A and a bifurcated distal end 7 with diverging forks 8A, 8B each terminated in a distal foot 9A, 9B. In the embodiment shown, the feet 9A, 9B are laterally spaced apart by about 7 cm, although it will be appreciated that the spacing may be varied according to the bone being treated and the type of fracture. Each foot 9A, 9B is formed of rubber and has a frusto-conical shape with a height of about 1.5 cm and a diameter at it base of about 2.5 cm.

The second arm 3 has a proximal section 10 with a handle 6B and a distal end 11 terminated in a bone engaging jaw 13. The bone-engaging jaw is counter opposed but offset to the feet 9A, 9B, being counter-opposed to a position midway between the feet. The bone engaging surface of the jaws is slightly arcuate to extend across a surface of the bone and comprises a series of serrated teeth 25 to facilitate the forceps grasping the bone.

The pivot joint 4 is a conventional pivot joint used in orthopaedic forceps and will not be described in more detail.

The forceps 1 also includes a ratcheting mechanism comprising counterfacing ratcheting racks 15A, 15B to lock the two arms, relative to each other, and, thereby, maintain a force between the jaws of each arm to hold the bone fragments together after the forceps have been released from a surgeon's hand. Elastic deformation of the arms generally provides the force.

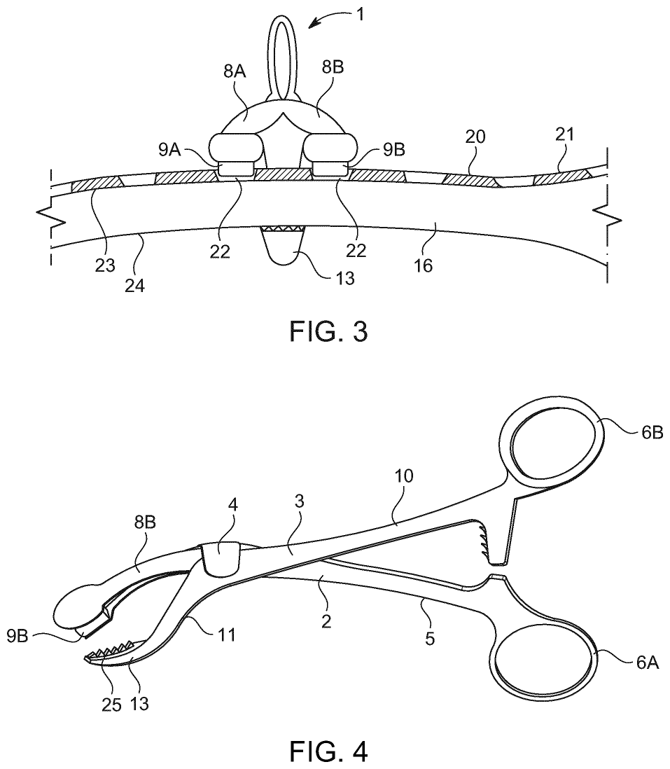

Referring to FIG. 3, a bone reduction kit according to the invention is described in use plating a human clavicle 16 in a reduced position. The kit comprises the bone reduction forceps 1 described with reference to FIGS. 1 and 2, and a bone fixation plate 20 which in this case is a contoured clavicle plate. The plate 20 comprises an obverse face 21 with a plurality of countersunk holes 22 having a frusto-conial shape that matches the shape of the rubber distal feet 9A, 9B. The plate 20 is placed on a superior surface 23 of the clavicle with the contoured surface of the plate 20 matching the contours of the superior surface of the clavicle. The forceps is then aligned with the plate 20 and clavicle 16, with the distal feet 9A, 9B aligned with the countersunk holes 22 and the bone-engaging jaw 13 positioned below an inferior surface 24 of the clavicle counter-opposed to a position between the distal feet. Actuation of the forceps results in the rubber feet 9A, 9B mating with the countersunk holes 22 and the jaw 13 engaging the inferior surface of the clavicle, with the result that the plate is held securely in position on the reduced clavicle. The forceps may be locked in this position using the ratcheting racks 15A, 15B allowing the surgeon attached the plate to the clavicle.

The forceps of the invention may be employed to hold bone fragments in a reduced position and/or hold a plate in position on a bone while the plate is being fixed to the bone. It is particularly applicable for plating the clavicle, particularly mid-shaft plating of the clavicle.

The embodiments illustrated show a 3-point forceps (first arm is bifurcated and bears two bone-engaging jaws). However, it will be appreciated that the second arm may also be bifurcated and have two forks and two bone-engaging jaws (i.e. a 4-point forceps). Moreover, it will be appreciated that the first arm may include three of more forks and associated distal feet. A preferred embodiment is a 3-point forceps with two distal feet and one bone-engaging jaw. The jaw may be elongated along an axis of the bone so that it engages the bone along a length of the bone, for example 5—20%, 20-30%, 30-40%, 40-50% or 50-60% of the length of the bone.

### Equivalents

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A bone reduction kit to hold a bone fixation plate (20) in position on an at least party reduced bone (16) while the bone fixation plate is fixed to the bone, comprising:
a bone fixation plate (20) having a bone abutting surface and an obverse face (21) and a plurality of screw-receiving though-holes; and
a bone reduction forceps (1) comprising:
a first arm (2) having a proximal part (5) comprising a handle (6A) and a bifurcated distal part having a first fork (8A) with a first distal foot (9A) at a distal end of the first fork and a second fork (8B) with a second distal foot (9B) at a distal end of the second form;
a second arm (3) having a proximal part (10) comprising a handle (6B) and a distal part (11) comprising a bone engaging jaw (13) configured to engage a second surface (24) of the bone (16);
wherein the second arm is pivotally attached to the first arm by a first joint (4) disposed between the respective proximal and distal parts of the first and second arms;
characterized in that the obverse face (21) of the bone fixation plate (20) comprises two spaced-apart depressions (22) positioned and dimensioned to mate with first and second distal feet (9A, 9B) of the forceps whereby when the forceps is clamped on the partly reduced bone and plate, lateral movement of the plate with respect to the bone is prevented.

2. A bone reduction kit according to claim 1, in which the first and second distal feet (9A, 9B) comprises a resiliently deformable elastomeric material.

3. A bone reduction kit according to claim 2, in which the first and second distal feet (9A, 9B) comprise a rubber material.

4. A bone reduction kit according to any preceding Claim, in which at least one of the depressions (22) is a counter-sunk or counter-bored through-hole.

5. A bone reduction kit according to any preceding Claim, in which both of the depressions (22) are counter-sunk or counter-bored through-holes.

6. A bone reduction kit according to any preceding Claim, in which the first and second distal feet (9A, 9B) have a frustum shape.

7. A bone reduction kit according to any preceding Claim, in which the first and second distal feet (9A, 9B) have a frusto-conical shape.

8. A bone reduction kit according to claim 6, in which the depressions have a frustum shape that matches the frustum shape of the respective first and second distal feet.

9. A bone reduction kit according to claim 8, in which the depressions have a frusto-conical shape that matches the frusto-conical shape of the respective first and second distal feet.

10. reduction kit according to claim 3, in which the first and second distal feet are positioned so as to engage a middle third of the bone fixation plate (20) when the forceps is clamped on the partly reduced bone and plate.

11. A bone reduction kit according to any preceding Claim, in which the bone engaging jaw is counter-opposed to a position in between the first and second distal feet.

12. A bone reduction kit according to any preceding Claim, in which the bone engaging jaw (13) comprises a plurality of teeth (25).

13. A bone reduction kit according to any preceding Claim, including a ratcheting mechanism (15A, 15B) attached to one of the first and second arms.

14. A bone reduction kit according to any preceding Claim, in which the bone fixation plate (20) is a clavicle fixation plate configured to conform to a superior (23) or inferior (24) surface of a reduced human clavicle (16).
